(19) United States
(12) Patent Application Publication
(10) Pub. No.: US 2023/0052806 A1
(43) Pub. Date: Feb. 16, 2023

(54) METHODS AND APPARATUSES FOR DELIVERY OF AN AGENT TO THE LUNGS AND NASAL PASSAGES

(71) Applicant: Inhalon Biopharma, Inc., Carrboro, NC (US)

(72) Inventors: Samuel K. LAI, Durham, NC (US); Keiichiro KUSHIRO, Durham, NC (US); Thomas R. MOENCH, Baltimore, MD (US)

(21) Appl. No.: 17/889,141

(22) Filed: Aug. 16, 2022

Related U.S. Application Data
(60) Provisional application No. 63/233,661, filed on Aug. 16, 2021.

(51) Int. Cl. A61M 15/00; A61K 9/00; C07K 16/00

(52) U.S. Cl. CPC A61M 15/009; A61M 15/0021; A61K 9/0078; C07K 16/00; C07K 2317/24; C07K 2317/52

(57) ABSTRACT

Methods and apparatuses (e.g., systems, devices, etc.) for delivering a nebulized drug agent in the nasal passages concurrent with the lungs.

### CROSS REFERENCE TO RELATED APPLICATIONS

This application claims priority to U.S. Provisional Patent Application No. 63/233,661, titled “METHODS AND APPARATUSES FOR DELIVERY OF AN AGENT TO THE LUNGS AND NASAL PASSAGES” and filed on Aug. 16, 2021, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

The expiratory phase of normal human respiration typically lasts approximately twice as long as the inspiratory phase. Most nebulizers, including vibrating disk nebulizers, produce nebulized droplets continuously, because timing aerosol production according to the phase of respiration adds complexity and a cost. Only the nebulized material available and produced during the inspiratory phase enters the patient's respiratory tract; nebulized material produced during the expiratory phase is wasted.

It would be desirable and advantageous to provide a method to administer nebulization treatments whereby a greater fraction of the treatment reaches the patient, and a smaller fraction is wasted. Furthermore, it would be desirable to provide methods and apparatuses for delivering a significant amount of treatment (e.g., therapeutic agent) to both the upper respiratory tract (e.g., nasal passages) and the lower respiratory tract (e.g., lungs) with a smaller amount of therapeutic agent, and to provide appropriate balanced delivery to the upper and lower respiratory tract. The surface area available in the nasal mucosa is estimated to be about 180-200 cm², of which 10 cm² is olfactory mucosa and remaining the richly vascularized respiratory mucosa. In contrast, the lower respiratory tract (comprising the trachea, bronchi, bronchioles, and alveoli) has a collective surface area of 140 m², of which the upper tracheobronchial and lower bronchial surface area is roughly 2,350 cm².

### SUMMARY OF THE DISCLOSURE

Described herein are methods and apparatuses (e.g., systems, devices, etc.) for delivering a nebulized drug agent in the nasal passages, including delivering a significant amount drug agent into both the nasal passages and the lungs. In general, these methods and apparatuses may include or be used with a continuous nebulizer and may instruct, assist or compel the patient to repeatedly inhale the nebulized drug agent (e.g., “drug”, and in particular drug agents that bind to mucus) through the mouth and exhale through the nose, and inhale the nebulized drug agent for a longer period than exhale. For example, the patient may inhale for four (4) or more seconds, and exhale for 3 or fewer seconds (e.g., 2 or fewer seconds). Surprisingly, these methods and apparatuses may result in a significant deposition of the drug agent in the nasal passages in addition to the lower respiratory tract (e.g., lungs). The surface concentration of the drug agent between the nose and the lung may comport closely with the relative areas of the nasal passages relative to the lung. In particular, the amount (e.g., concentration) of drug agent in the nasal passages may be many times the typical IC50 for the nebulized drug agent(s); in some examples the concentration of drug agent in the nasal passages may be many hundreds or thousands of times the IC50 of the drug agent(s). These methods may also enable more effective delivery to the nasopharynx.

Thus, the methods and apparatuses described herein may maximize delivery of a drug agent to the lower respiratory tract (e.g., lungs) while also delivering drug agent to the nasal passages in a manner that achieves a relatively high effective concentration in the nasal lining. The amount of drug agent in inhaled nebulized particles deposited in the nasal passages (e.g., nasal lining) may be adequate for the therapeutic purpose, while still depositing greater quantities to the lung, which has a far greater surface area and consequently a proportionally greater volume of fluid lining, thereby leading to a more appropriate balance to these two areas, and achieving a more uniform surface concentration in the lungs and the nasal passages.

The amount of air moved during quiet respiration (the tidal volume), is typically smaller than the maximum amount of air that can be inspired with effort to fully inflate the lungs. Thus, quiet respiration does not fully inflate the lung, and may leave certain portions of the lung only minimally inflated, poorly supplied with air, and therefore poorly supplied with nebulized drug. The methods and apparatuses described herein may provide nebulization treatments to a higher proportion of the lung than is reached by quiet respiration at the tidal volume. Deep inspiration has the advantages listed above, but the rate of the inspiration should be slow rather than rapid, since high rates result in loss of aerosolized droplets by inertial droplet collisions around the larynx and other bends in the respiratory tract.

Respiratory infections often infect tissues of both the upper airway (nasal passages) and lower airways (in the lung), and occasionally even cells in the oral mucosal. Medications to treat these infections may be administered directly to the respiratory tract by nebulization, and to be maximally effective, should treat both the upper and lower airways as well as the oral mucosa. Nebulizations can be administered through a mask covering the mouth and nose, allowing inspiration through both the mouth and nose. But since the surface area of the nasal passages is many orders of magnitude smaller than the surface area of the airways of the lung, the fraction entering and deposited in the nasal passages may be excessive for what would be needed for adequate treatment of that small surface area, while consequently greatly reducing the fraction of the treatment that can reach the lower respiratory tract. The nasal passages may effectively filter inhaled nebulized droplets. Conversely, inadequate treatment may be given to the nasal passages because patients may inhale almost entirely through the mouth even when using a mask, and may be unable to assure that any air is entering the nose. Moreover, masks can be uncomfortable, or can induce a claustrophobic sensation, and can lead to excessive deposition and moisture accumulation on the surrounding skin. As a result, many patients prefer to inhale using a mouthpiece instead of a mask. This method of administration may provide no treatment to the nose on inspiration. It would be desirable and advantageous to provide a method to administer nebulization treatments where it can be assured that the drug being administered is certain to reach the nasal passages, and whereby the amount of the drug between the nose and the lung approximately comports with the fractional area of the nasal passages relative to the lung. In some examples, surface concentration may be approximately the same between the nasal passages and the lungs.

Without being bound by theory, the rapid exhalation following a longer inhalation may result in a significant deposition of nebulized particles as compared to longer, slower exhalation. The nasal turbinates are structures that protrude into the nasal passages and create turbulence in the inspired air stream. This turbulence results in capture of inhaled environmental particulates by causing inertial impaction of particles on the nasal mucosal surfaces, thus protecting the lung from exposure to potentially harmful particles. Medical treatments may be given to the respiratory tract by production of drug-agent-containing droplets by nebulizer devices, and inhalation of the resultant aerosols to treat the respiratory tract.

Nebulization treatments administered by inhaling through the nose typically results in extensive nasal deposition of nebulized droplets because nasal turbinates act on these droplets similarly to the way they act on and capture inhaled environmental particles. This causes the majority of nasally inspired droplets to be delivered to the nose, and therefore may result an inadequate quantity of droplets delivered to the lower airways. Delivering the majority of the inhaled dose to the nose is particularly disadvantageous when it is considered that the surface area of the nasal lining is very small compared to the area of the lower respiratory tract. The surface area of the nasal passages is estimated at less than 0.02 square meters, and the surface area of the lower respiratory tract is estimated at approximately 100 square meters. Thus the surface area lung is between three and four orders of magnitude larger than the surface area of the nasal passages. Therefore it is wasteful and potentially ineffective to deliver a substantial portion of a nebulized drug via nasal inspiration, since this will overtreat the nose, and undertreat the lung. Inadequate delivery to the lungs can be avoided by inhaling the nebulization treatment via the mouth. However, for the treatment of respiratory infections, it is necessary and advantageous to provide treatment to both the lower respiratory tract, and the nasal passages. For example, many respiratory tract pathogens can replicate in both the lower respiratory tract, oral mucosa and in the nasal passages. Thus both the nose and the lung must be treated, but for efficiency and full efficacy in the lower respiratory tract, excessive treatment of the nose should be avoided.

Nebulization treatments may be administered using a mask that covers both the mouth and the nose, e.g., using a mask, and may simultaneously treat both the nasal passages and the lung. However, because there is little or no control over relative amount of air entering through the mouth versus the nose, a substantial amount may enter via the nose, resulting in significantly waste due to overtreating the nose, and possibly undertreating the lung. Similarly, inhaling and exhaling exclusively through the mouth (e.g., using a mouthpiece and/or blocking the nose), may result in a high efficiency in providing drug agent to the lower respiratory tract, but this method would not provide treatment to the nasal passages.

The methods and apparatuses described herein may provide efficient treatment of both the lower respiratory tract (e.g., the lungs) and the upper respiratory tract (e.g., the nasal passages). The amount of drug agent deposited in the nasal passages may be commensurate with the surface area of the nasal passages. Moreover, the treatment of the nasal passages may be achieved by capturing in the nose those nebulized droplets suspended in the air filling the dead space of the large airways at end inspiration, which would otherwise be expelled via the mouth and wasted.

For example, described herein are methods of delivering particles of a drug agent to a patient's nasal passages (or to a patient's lungs and nasal passages), that include: operating a nebulizer containing the drug agent to continuously form particles containing the drug agent; holding a mouthpiece of the nebulizer in the mouth with lips sealed over the mouthpiece; and repeatedly: inhaling for 4 seconds or longer from the nebulizer through the mouth followed by exhaling only through the nose for 3 seconds or less. The nebulized particles (including the drug agent) may thus be delivered to the nasal passages and the lungs.

Any of these methods may also be methods of achieving an approximately equivalent surface concentration of the drug in the nasal passages and lower respiratory tract (e.g., lungs) of the patient. The amount of drug agent deposited in the nasal passages may be many times the 50% inhibitory concentration (IC50) of the drug agent (e.g., more than 100×, more than 1000×, etc.).

As used herein, the term “nasal passages” may include all or a portion of the upper respiratory tract, such as the nose, nasal cavity (including nasal mucosa). The nasal passages may include the choanae (openings between the nasal cavities and the nasopharynx), the vestibule, nasal fossae (superior, middle, and inferior nasal conchae), and the superior, middle and inferior nasal meatus.

As used herein, the term “lower respiratory tract” may refer to the lungs, trachea, bronchi, and/or alveoli. Any of the methods and apparatuses described herein may be used for delivering drug agent to one or more region of the lower respiratory tract, and in particular, the lungs, so as to achieve an approximately equivalent surface concentration as compared to the nasal passages.

As used herein an approximately equivalent surface concentration may refer to the relative concentrations of the nebulized droplets and/or the active agent in the nebulized droplets that are present, e.g., between the lower respiratory tract (e.g., lungs) and nasal passages.

Any of these methods may include inhaling for 4 seconds or longer, including but not limited to inhaling for between 4-10 seconds (e.g., 4.5 seconds or longer, for 5 seconds or longer, for 6 seconds or longer, for 7 seconds or longer, etc.). Exhaling only through the nose for 3 seconds or less may include exhaling only through the nose for 2 seconds or less (e.g., between 0.2 seconds and 3 seconds, between 0.5 seconds and 2.5 seconds, etc.). The cycle of deep inhalation (4 seconds or more) followed by rapid exhalation (3 seconds or less) may be repeated 1 or more times (e.g., 2 or more times, 3 or more times, 4 or more times, 5 or more times, 6 or more times, etc.). The cycle may be followed by a period in which normal breathing is performed (with the nebulizer) and then repeated again one or more times.

Any of the methods described herein may be performed with a nebulizer and/or guide apparatus (or the nebulizer may include or be incorporated into guide apparatus). For example, the nebulizer or a separate dose guide apparatus may be used to guide the patient in inhaling the nebulized drug agent and exhalating in the required durations and/or patterns. For example, the nebulizer and/or separate dose guide apparatus may include indicators for indicating to the patient when and/or for how long (e.g., 4 or more seconds) to inhale and then when and/or for how long (e.g., 3 seconds or less) to exhale. For example, the apparatus (e.g., nebulizer and/or guide apparatus) may indicate with a first indicator when and/or for how long to inhale the nebulizer followed indicating with a second indicator when and/or for how long to exhale. The first and/or second indicators may be a visual indicator (e.g., light, LEG, display, etc.) and/or an audible indicator (tone, beep, countdown, etc.) and/or a tactile indicator (e.g., vibration). Any of these apparatuses may include a trigger that is manually or automatically triggered by the start of inhalation. The apparatus may detect inhalation (negative pressure) applied by the patient and may trigger the first indicator to provide immediate feedback to the patient to continue inhaling for at least the minimum duration (e.g., 4 or more seconds). The apparatus may detect exhalation and/or may simply alter the patient to exhale for the duration of the second indicator (e.g., exhale for 3 or less seconds), repeating the processes to continue dosing for the desired (predetermined) dose.

Thus, in any of these methods the method may include triggering a first indicator for the period of inhalation, wherein the first indicator is triggered for 4 seconds or longer and triggering a second indicator for the period of exhalation wherein the second indicator is triggered for 3 seconds or less.

In some examples the nebulizer and/or and dose guide apparatus may include software, hardware and/or firmware for indication (using the first and second indicators) when and/or for how long to inhale and exhale. In some examples the dose guide apparatus is separate from the nebulizer and may be operated with any nebulizer (particularly continuous nebulizers). For example, the does guide apparatus may be an application software that runs on a patient's phone or handheld (and/or worn) computing device. The first and second indicators may be provided by the phone and/or handheld (or worn) computing device executing the dose guide apparatus application software. For example the first indicator may be a display and/or tone emitted by the patient's (or caregiver's) phone. In some examples the nebulizer is integrated with the dose guide apparatus. For example the nebulizer may include one or more outputs (LEDS, displays, speakers, etc.) to provide the first and second indicators. In some examples the nebulizer may include one or more sensors for detecting inhalation and/or exhalation.

In general any appropriate nebulizer may be used, but in particular a nebulizer having a mouthpiece that can be held between the patient's lips. These apparatuses may be continuous nebulizers, such as vibrating mesh nebulizers, and may continuously operate to nebulize the drug agent.

The nebulizers described herein may be configured to generate particle sizes with a predetermined range. The particle size range may be within a preferred range for deposition within both the lungs and the nasal passages using the methods described herein. Particles outside of the desired range may not be delivered within the nasal passages with the desired distribution pattern or level. For example, in any of the methods described herein, operating the nebulizer to continuously form particles containing the agent may comprise forming particles of average particle or droplet size (commonly defined as median mass aerodynamic diameter, MMAD) in the range from about 0.1 to about 200 microns (such as between about 1 to 10 microns, between about 2 to 7 microns, between about 2 to 20 microns, between about 10-40 microns, between about 20-60 microns, between about 30-70 microns, between about 40-80 microns, between about 50-90 microns, between about 60-100 microns, between about 70-110 microns, between about 80-120 microns, between about 90-130 microns, between about 100-150 microns, between about 125-200 microns, etc.). For example, operating the nebulizer to continuously form particles containing the agent comprises forming particles of average particle or droplet size in the range from about 2 to 7 microns.

A variety of drug agents may benefit from the nebulized delivery methods and apparatuses described herein. In particular, these methods and apparatuses may benefit drug agents for treating respiratory disorders effecting both the upper and lower respiratory tracts. These methods and apparatuses may be particularly effective in delivery drug agents that are configured as mucosal binding and/or trapping agents. For example, the methods and apparatuses described herein may be particularly useful and/or effective when the drug agent is a recombinant antibody comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus. In some examples the drug agent comprises a recombinant antibody comprising a human or humanized Fc region, wherein the recombinant antibody comprises a population of antibodies in which at least 20% (e.g., 20% or more, 25% or more, 30% or more, 35% or more, 40% or more, etc.) comprise an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

For example, a method of delivering particles of a drug agent to a patient's nasal passages (or to both the patient's nasal passages and lungs in an approximately equivalent surface concentration of the drug between the nose and lungs of the patient), comprising: operating a nebulizer containing the drug agent to continuously form particles containing the drug agent; holding a mouthpiece of the nebulizer in the mouth with lips sealed over the mouthpiece; and repeating one or more cycles of: indicating, with a first indicator, an inhalation period of 4 second or longer, to guide the patient in inhaling particles containing the drug agent through the mouth for the inhalation period, and indicating with a second indicator, an exhalation period of 3 seconds or shorter, to guide the patient in exhaling through the nose but not the mouth for the exhalation period, wherein a significant concentration of the drug agent is deposited in the nasal passages.

Any of these methods may include triggering the first indicator at a start of inhalation by the patient. The triggering of the first indicator may be manual or automatic. In some examples the apparatus (e.g., nebulizer and/or dose guide apparatus) may detect, by receiving from a sensor such as a pressure or flow sensor, the start of inhalation and may trigger the indicator (which may count down the duration of inhalation, e.g. 4 seconds or more). In some examples the apparatus may include a button or other input that is triggered by the patient manually at the start of inhalation. In some examples the apparatus may start the indicator so that the patient may follow along.

Any of these methods may include triggering the second indicator at a start of exhalation by the patient. The second indicator may be manually or automatically triggered. For example the apparatus may detect, by receiving input from the same or a different sensor detecting inhalation that inhalation has stopped and/or that exhalation has begun. The second indicator may be manually triggered by the patient when stopping inhalation through the mouth. In some examples the apparatus may stop the first indicator and start the second indicator without any input from the patient or sensor(s), providing guidance and allowing the patient to follow along.

As mentioned, any appropriate one or more indicators may be used for the first and second indicators; generally the first indicator may be different from the second indicator. In some examples the first indicator may comprises illuminating a first LED and/or the second indicator comprises illuminating a second LED.

In general, these methods may include depositing nebulized particles of drug agent in both the patient's nasal passages and lungs in an approximately equivalent surface concentration of the drug agent between the nose and lungs of the patient. In general, the absolute amount of drug agent deposited in the lower respiratory tract (e.g., lungs) may be much larger than that deposited in the nasal passages, but, given the much larger surface area of the lungs, the proportion of the nebulized particles of drug agent deposited between the two may be approximately equivalent. As used herein, approximately equivalent may be within +/−about 10 fold (e.g., within +/−8 fold, +/−7 fold, +/−5 fold, +/−4 fold, +/−3 fold, +/−2 fold, +/−1.5 fold, etc.). For example, an approximately equivalent surface concentration of the particles containing the drug agent may include a surface concentration of between about 10:1 and 1:10 (e.g., between about 8:1 and 1:8, between about 5:1 and 1:5, between about 3:1 and 1:3, between about 2:1 and 1:2, between about 1.5:1 and 1:1.5, between about 1.2:1 and 1:1.2, etc.) between the patient's nasal passages and lungs.

In any of these methods and apparatuses, the inhalational period is between 4-10 seconds. In some examples the exhalation period is 2 seconds or less. Any of these methods may include operating the nebulizer to continuously form particles containing the agent comprises forming particles of average particle or droplet size in the range from about 0.1 to about 200 microns (e.g., between 2 to 7 microns, etc.), as described above.

In any of these methods the drug agent may be a recombinant antibody comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus. For example, the drug agent may comprise a recombinant antibody comprising a human or humanized Fc region, wherein the recombinant antibody comprises a population of antibodies in which at least 20% (e.g., at least 25%, at least 30%, at least 35%, at least 40%, etc.) comprise an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a schematic illustrating one example of a method as described herein.

FIG. 2 is a table (Table 1) showing the concentration of a sample drug agent (e.g., an antibody, shown in in units of micrograms per mL) in nasal secretions collected by swabbing a patient's mid-nasal turbinate after inhalation through the mouth and exhaling through the nose as described herein. Three patients were examined for between 3 and 5 trials.

FIG. 3A illustrates an example of a clinical trial process flow, and FIG. 3B illustrates an example of a study structure and times of sample collection for pharmacokinetic evaluations.

FIG. 4 is a table (Table 2) showing participant demographics for the study of FIGS. 3A-3B.

FIG. 5 is a table (Table 3) showing treatment emergent adverse events (TEAEs) for an example of the study of FIGS. 3A-3B.

FIG. 6 is a table (Table 4) showing results from the study of FIGS. 3A-3B for single dose treatments.

FIG. 7 is a table (Table 5) showing results of mean serum (% CV) of the study of FIGS. 3A-3B. In Table 5, n=2 for the samples (**). For Cohort 3, the value of 257,170 represents the AUC calculated through tₗₐₛₜ, to allow for direct comparison to Cohorts 1 and 2. However, since Cohort 3 had more quantifiable samples at later timepoints, AUC was also calculated until the serum concentration theoretically hit zero (AUC__{infinity_obs}), which was 404,034 hr*ng/mL, with % CV of 35%. The AUC_infinity was not calculated for Cohorts 1 and 2 because too many samples were BLQ at the later timepoints, making the estimate for the terminal phase of elimination less reliable.

FIG. 8A-8B are graphs showing the nasal fluid concentrations from the study described in FIGS. 3A-3B. FIG. 8A shows the nasal fluid concentration in single dose cohorts. The dotted line represents the average LLOQ, and symbols shown below that line represent the number of samples that fell below the LLOQ at that time. FIG. 8B shows a comparison of nasal concentrations between single dose and multiple dose cohorts at varying times after each dose. Average LLOQ for all nasal fluid samples is shown at 450 ng/g, but LLOQ varied by sample, depending on the mass of nasal fluid collected on swab.

FIGS. 9A-9B illustrate serum concentrations from the experiment described in FIGS. 3A-3B. FIG. 9A is a graph of the single dose cohorts, and FIG. 9B is a graph of the multiple dose cohort (last dose administered at 144 h). Symbols plotted below the dashed LLOQ line at 25 ng/mL represent the number of samples in each group that were BLQ at each timepoint.

### DETAILED DESCRIPTION

Most nebulizers, including vibrating mesh nebulizers release nebulized particles created by aerosolization of the liquid medication placed in the nebulizer (visible as a mist or aerosol) from the mouthpiece as soon as the device is switched on, and continue until the device is switched off. Mist created by the continuing action of the vibrating mesh may be lost to the outside environment before being inhaled, or during an exhalation. The drug contained therein never reaches the patient and is wasted. Based on medical literature descriptions of the ratio of expiration time to inspiration time, it is estimated that about two thirds of the medication will be wasted by being aerosolized and lost to the room during expiration. After breathing in the aerosol via the mouthpiece, moving the nebulizer to one side, and breathing out through the mouth, visible mist is apparent during exhalation, representing aerosol that was taken into the airways, and exhaled without being deposited against airway surfaces.

As described in greater detail herein, in some cases, nebulized drug agent may be inhaled into through just the mouth for a long period of time, and rapidly exhaled out of the nose (but not the mouth), no visible mist is observed exiting the nose. This demonstrates that some of the aerosol exiting the lungs on expiration is trapped in the nose during exhalation through the nose. Furthermore, increased sensation of fluid in the nose may be detected during exhalation through the nose, again demonstrating nasal deposition. Finally, as described herein, substantial nebulized drug concentration accumulates in the nose when exhaling through the nose during exhalation.

However, patients find it difficult or impossible to breath in the very counterintuitive mouth inhalation/nasal exhalation pattern described herein. In particular, the rapid (shorter than the deep inhalation) is counterintuitive, particularly when targeting nasal passages. Surprisingly, the methods and apparatuses described herein allow an approximately equivalent relative surface concentration of the drug between the nose and lungs of the patient even when counterintuitively inhaling exclusively through the mouth and exhaling for less than the duration of inhalation.

As described herein, the loss of drug due to ongoing aerosolization during expiration can be substantially reduced by having the patient use the nebulizer while prolonging their inhalation phase and shorten their exhalation phase. Initial data from four individuals, showed that these patients were able to extend the duration of inhalation to at least four seconds, with some able to extend inhalation to seven seconds. All four individuals were able to shorten exhalation to less than 3 seconds, and in some cases shortened exhalation to approximately 2 seconds. Individuals were passively guided (e.g., instructed) to inhale deeply for 4 seconds or longer and to exhale more rapidly (e.g., for 3 seconds or less). Thus, the ratio of inhalation to exhalation was voluntarily increased to 4:3 in all individuals, and to 7 to 2 in some individuals. In contrast, the reported ratio of inhalation to exhalation when breathing according to natural breathing patterns is approximately 1:2, even when longer inhalation is taken. Preliminary data shows that either passive or active guidance in prolonging the inspiratory phase and shortening the exhalation phase resulted in a substantial reduction in waste due to ongoing nebulization during exhalation, and significant deposition in the nasal passages.

For example, subjects (e.g., patients) were instructed to inhale to the fullest extent possible using a slow rate of inspiration, and continuing inhalation until the lungs were completely full. This may result in distribution of the aerosol to deeper portions of the lung, to avoid loss of drug at the larynx, and may also result in treatment of areas of the lung that would not fully inflate without maximum inspiration. Rapid exhalation also results in maintaining the distribution of particles within the lungs, and also significant distribution of particles within the nasal passages. Most critically, an approximately equivalent surface concentration of the drug agent was found between the nasal passages and lower respiratory tract (e.g. lungs) of the patient. The fraction distribution in this context refers to the proportion of the nebulized particles of drug agent that were deposited, comparing the two regions (or parts of the two regions).

In general the methods described herein may include breathing into the nebulizer mouthpiece (which may, in particular, be a continuously nebulizing device), slowly for 4 seconds or more, e.g., over 4 seconds, over 5 seconds, over 6 seconds, etc. including between 4 to 7 seconds, etc., until the lungs are filled to a maximum extent; the patient may then breath out through the nose as rapidly as possible. Specifically, these methods may include guiding the patient in breathing in this pattern. As used herein, guiding may include instructing, showing, or the like, and may be passive (e.g., triggering an alert, display, tone, etc.) or active (closing/opening, in the nebulizer and/or a dose guide apparatus, one or more valves to enforce the breathing pattern when using the nebulizer. In some examples, the valve may be a one-way valve, such as a flap valve, ball valve, or other check valve that prevents or limits exhalation through the mouthpiece. Any of these methods may include instructing or guiding the patient to perform the method of inhaling through the mouth from the nebulizer for 4+ second and exhaling rapid (for less than 3 seconds) from the nose for one or more cycles.

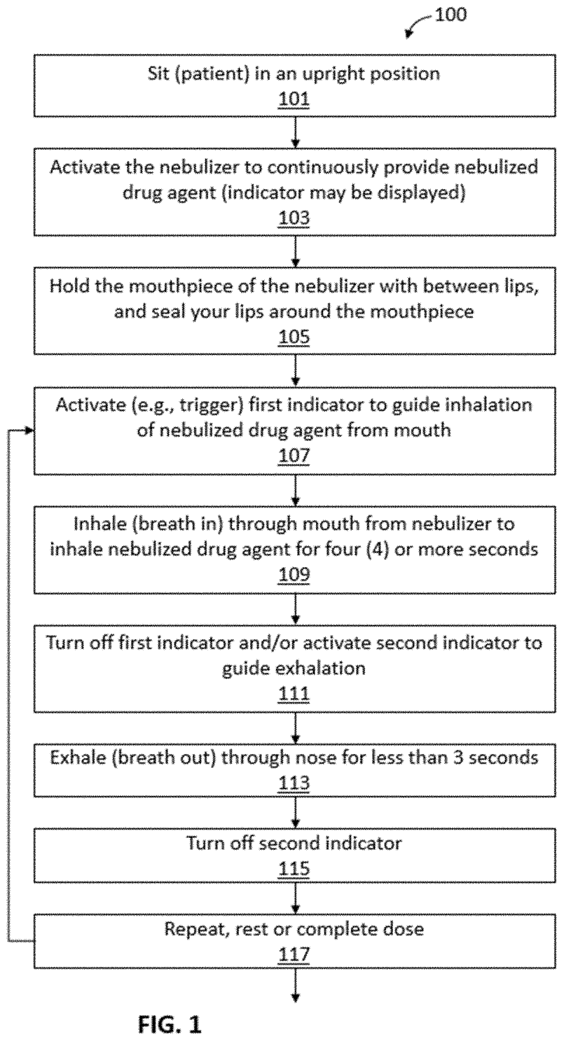

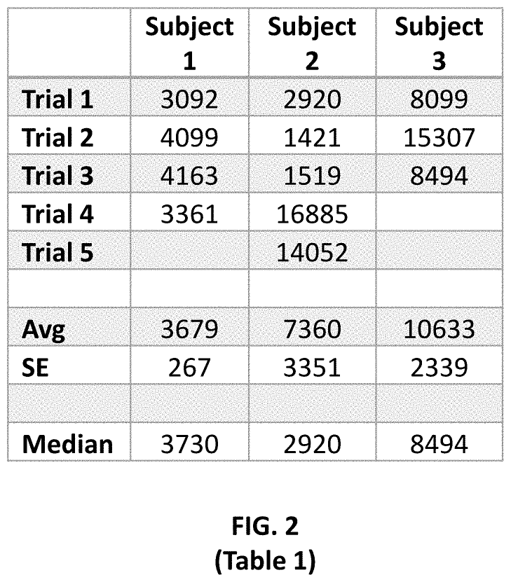

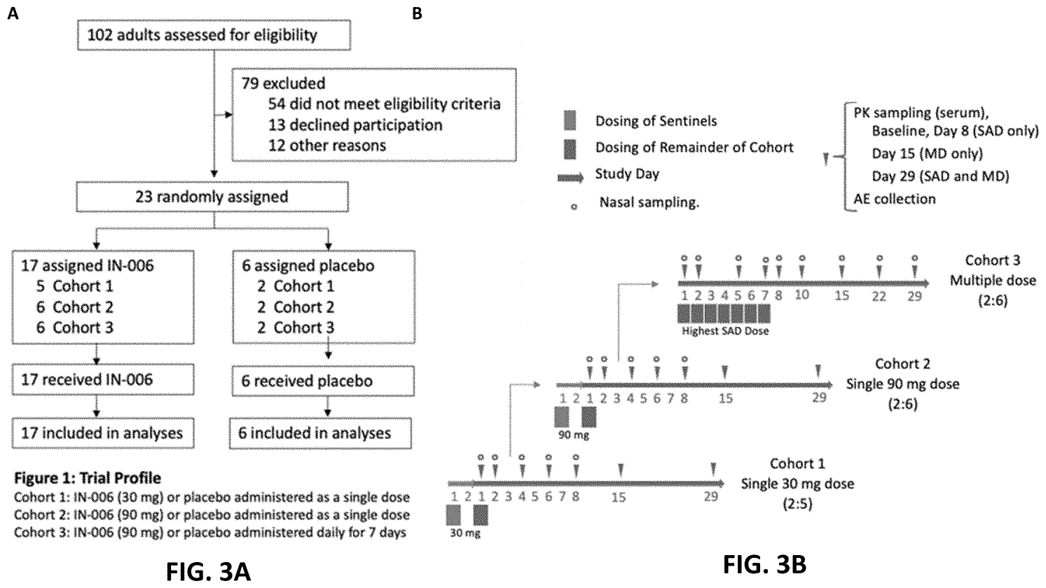

In one example, shown by the schematic in FIG. 1 the method 100 may include instructing and/or guiding the patient to administer a nebulization dose by first (optionally) sitting in an upright position 101. The patient may then activate the nebulizer to continuously provide a nebulized drug agent 103. For example, the method may include guiding the patient to press the on/off button on the nebulizer to start the treatment (e.g., in some examples the button will turn green, and mist will appear at the mouthpiece and/or the back of the nebulizer). The method may then guide the patient to hold the mouthpiece of the nebulizer between the lips 105, including holding the mouthpiece with the teeth and/or lips, and sealing the lips around the mouthpiece.

The method may then include activating (e.g., triggering) a first indicator to coach or guide the patient in inhaling the nebulized drug agent through the mouth 107. The first indicator may be, for example, a light (LED or LEDs), tone, message, countdown, etc. that remains on while the patient inhales to guide them to inhale deeply to draw the nebulized agent in through the mouth. The indicator may be a count (e.g., counting up or down). The indicator may be triggered automatically, including by a controller with or without input from the patient. In some examples the patient may manually trigger the start (activation) of the first indicator. Alternatively, in some examples the first indicator may be triggered upon sensing (e.g., in the nebulizer and/or in a dose guide apparatus) that the patient has started inhaling through their mouth. The first indicator may remain on for the inhalation duration of, e.g., 4 seconds or more (e.g., 4 seconds, 4.5 seconds, 5 seconds, 6 seconds, 7 seconds, etc.). The inhalation duration may be fixed or set (e.g., by a user, such as the physician, nurse, pharmacist, and/or the patient) or it may be variable. In some examples the inhalation duration may change to indicate that the minimum inhalation duration (e.g., of four seconds, 4.5 seconds, 5 seconds, etc.) has been reached, but that continuing inhalation is recommended. For example, the first indicator may be active for a minimum inhalation duration of 4 seconds using a first tone, color, etc., and may remain on for another 2-3 seconds but may change to a first optional/continuing indicator using a second tone, color, etc. For example, the nebulizer and/or dose guide apparatus may change from a red color to a yellow color or some other change to indicate inhalation may optionally continue.

The first indication (and/or the first optional/continuing indicator) may turn off automatically, e.g., after the patient has finished inhaling through the nebulizer and/or begun exhaling. The methods and apparatuses may include sensing inhalation and/or exhalation. For example the nebulizer and/or dose guide apparatus may include one or more sensors for detecting or deducing the start/stop of inhalation and/or exhalation. For example, a nebulizer may include one or more sensors for detecting flow or pressure at the mouthpiece. A flow sensor may be used to determine the start and/or stopping of inhalation through the mouthpiece. Any of these methods and apparatuses may include a controller (including one or more processors) that may perform these methods including triggering the first indicator, second indicator, etc.). The controller may analyze the sensor data to trigger the first and/or second indicators.

In general, the methods described herein may include instructing or guiding the patient to breathe in so that each breath is slow and long, breathing in until their lungs are as full as possible (e.g., breathe in as deeply as possible). Each inward breath in should last at least 4 seconds or longer as mentioned 109.

The second indicator, guiding the patient for the rapid (e.g., 3 seconds or less) exhalation may be triggered automatically as mentioned above (e.g., at the stop of inhalation) or based on a preset and/or settable timer. Generally, the method may include turning off the first indicator and/or activating the second indicator to guide exhalation 111. Because the exhalation phase is intended to be rapid and brief, the second indicator may include a “stop” indicator after the second (exhalation) duration of 3 seconds or less (e.g., 2 seconds), to alert the user to stop. For example, in some cases the second indicator may include a first phase from the start of exhalation to the end of the exhalation phase (2-3 seconds) 113 after which the second indicator may change to emphasize that the exhalation should be complete, for example by a change in the volume, tone, intensity, color, continuity (e.g., flashing) or the like. The second indicator may then turn off or otherwise stop 115.

Thus, during inhalation, the patient may be instructed and/or guided to breath out quickly through their nose, trying to finish breathing out within about 3 seconds (within about 2 seconds, within about 2-3 seconds, etc.). As discussed herein, this may direct the nebulized drug agent (e.g., mist) from the patient's lungs in the nose in the desired distribution, where it may be captured and give treatment to this area.

After the completion of the long inhalation/rapid exhalation, the patient may be instructed to either rest, e.g., breathe normally for one or more breaths, without the nebulizer, or to perform another cycle of long inhalation/rapid exhalation 117. For example, the patient may need to take a rest or if they have a cough or urge to cough. The patient may press the on/off button to stop the nebulizer. The treatment may be continued by once again pressing the on/off button on the nebulizer and/or dose guide apparatus to begin breathing in through the mouthpiece and out through the nose (repeating steps 107 to 117 in FIG. 1). The patient may take as many rests as needed.

Treatment may be continued until the desired (e.g., pre-set, user set, etc.) dose has been delivered. In some examples the treatment, including multiple cycles of long inhalation/rapid exhalation may be continued until the nebulizer and/or dose guide apparatus indicates the full treatment dose has been delivered. For example, the treatment may be continued until the nebulizer issues an alert (e.g., a beep and/or light flash), indicating that the treatment is complete. The device may turn off automatically.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

As mentioned above, an apparatus may be configured to perform any of the methods described herein. For example, an apparatus may be configured as a nebulizer integrated with a (or forming the) dose guide apparatus. The nebulizer may be configured to emit the first indicator, such as a tone (e.g., beeping, etc.) or illuminating one or more LEDs (e.g., a countdown of LEDs), and the second indicator, such as a second tone or illuminating a different color or set of LEDs, etc. As mentioned above, the nebulizer may include one or more sensors for detecting and triggering the start of inhalation and/or exhalation to allow the device to count down and guide the user in inhaling and exhalating as described herein.

In some examples a separate dose guide apparatus may be used with a nebulizer. For example, the dose guide apparatus may be software. In some examples the software may be executed on a processor of a wearable or hand-held computing device, such as a smartphone.

These methods and apparatuses may be used with any type of nebulizer. For example, these apparatuses may be used with a jet nebulizer that uses a compressed gas to make an aerosol, an ultrasonic nebulizer, which forms the aerosol through high-frequency vibrations and/or a mesh nebulizer that passes liquid passes through a very fine mesh to form the aerosol. In particular, these methods may be used with continuous nebulizers that continuously form particles when on. Alternatively, these methods may be used with on-demand nebulizers.

In general, the methods and apparatuses described herein may apply to aerosol particles of a specific or predetermined size or size distribution. For example the particles of drug agent (MMAD) may be in the range from about 0.1 to about 200 microns (such as between about 1 to 10 microns, between about 2 to 7 microns, between about 2 to 20 microns, between about 10-40 microns, between about 20-60 microns, between about 30-70 microns, between about 40-80 microns, between about 50-90 microns, between about 60-100 microns, between about 70-110 microns, between about 80-120 microns, between about 90-130 microns, between about 100-150 microns, between about 125-200 microns, etc.). For example, particles containing the agent may have a particle or droplet size in the range from about 2 to 7 microns. In some examples the method described herein may be used with two distributions of particle sizes, including smaller and larger particle sizes.

Any appropriate drug agent may be used, including but not limited to drug agents that are mucosal trapping drug agents and/or immunotherapeutics. In general these drug agents may be drug agents for treating a respiratory disorder/disease, including disorders/diseases that are transmitted by respiration.

In particular, the drug agents described herein may include drug agents that are trapped within mucus, as described, e.g., in each of U.S. Pat. Nos. 10,829,543, 10,100,102, 10,793,623, U.S. patent application Ser. No. 16/982,682 (titled “COMPOSITIONS AND METHODS FOR INHIBITING PATHOGEN INFECTION” and filed Mar. 20, 2019), U.S. patent application Ser. No. 17/063,122 (titled “OPTIMIZED CROSSLINKERS FOR TRAPPING A TARGET ON A SUBSTRATE” and filed Oct. 5, 2020), and U.S. patent application Ser. No. 17/278,217 (titled “SYNTHETIC BINDING AGENTS FOR LIMITING PERMEATION THROUGH MUCUS” and filed Sep. 23, 2019), each of which is herein incorporated by reference in its entirety.

For example, the methods described herein may be particularly useful for delivering a dose of a drug agent that is configured to have an enhanced trapping potency in mucus, including but not limited to proteins (e.g., antibodies) that include one or more glycosylation patterns that enhance trapping in mucus. In some examples the drug agent may be a recombinant antibody comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus. For example, the drug agent may be a recombinant antibody comprising a human or humanized Fc region, wherein the recombinant antibody comprises a population of antibodies in which at least 40% comprise an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

### EXAMPLES

FIG. 2 is a table (Table 1) showing one example illustrating the concentration of antibody (in units of micrograms per mL) in nasal secretions collected by swabbing the patient's mid-nasal turbinate following inhalation from a test drug agent (antibody) that was nebulized using a Phillips Innospire Go nebulizer. Each subject (patient) was instructed to nebulize 4 mL of antibody at 25 mg/mL concentration by breathing in (i.e. inhale) through the mouthpiece without wearing a mask and was further instructed to breathe out (i.e. exhale) primarily through the nose. Five minutes after the completion of the nebulization, mid nasal turbinate swab(s) were collected, in most instances from both sides of the nasal passageway. The concentration of the antibody was then measured by ELISA and corrected for the dilution that occurred when recovering antibodies from the swab using 1 mL of buffer. For all subjects the oral inhalation of drug agent (antibody) when exhalating through the nose resulted in a significant concentration (micrograms per mL) of antibody in the nasal passage.

To place the results shown in FIG. 2 in perspective, the 50% inhibitory concentrations for select monoclonal antibodies against different respiratory viruses can be as little as 1-20 nanograms per mL. Thus, the data here demonstrates that these methods are capable of achieving substantial and therapeutically meaningful concentrations of drug agent (e.g., antibodies) in the nasal mucus lining by using the oral-inhale nasal-exhale method of nebulized delivery. Concentrations in the 3-10 milligrams represents a factor of 100,000 to 1,000,000 fold higher than the IC50 of the antibody in the nasal mucosa.

### Example 2—Randomized, Double-Blind Study of Spike Protein of SARS-CoV-2 Antibody

Although COVID-19 is predominantly a respiratory tract infection, current antibody treatments are administered by systemic dosing. Inhaled delivery of a muco-trapping monoclonal antibody may provide a more effective and convenient treatment for COVID-19. The methods and apparatuses described herein were used to deliver IN-006, a reformulation of regdanvimab, an approved intravenous treatment for COVID-19, for nebulized delivery by a handheld nebulizer as described herein. This therapeutic antibody is a human/humanized monoclonal antibody directed to the spike protein of SARS-CoV-2. This study was conducted in healthy volunteers. Study staff and participants were blinded to treatment assignment, except for pharmacy staff preparing the study drug. Pharmacokinetic measurements of IN-006 in nasal fluid and serum were examined. Twenty-three participants were enrolled and randomized across two single dose and one multiple dose cohorts. There were no serious adverse events (SAEs). All enrolled participants completed the study without treatment interruption or discontinuation. All treatment-emergent adverse events were transient, non-dose dependent, and were graded mild to moderate in severity. Nebulization was well tolerated and completed in a mean of 6 minutes in the high dose group. Mean nasal fluid concentrations of IN-006 in the multiple dose cohort were 921 μg/g of nasal fluid at 30 minutes after dosing and 5.4 μg/g at 22 hours. Mean serum levels in the multiple dose cohort peaked at 0.55 μg/mL at 3 days after the final dose.

The results with IN-006 described herein in detail are representative of results for other therapeutic antibodies delivered by nebulization described herein, including recombinant antibodies having an oligosaccharide with a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus. Delivery of the therapeutic mAb via inhalation as described herein shows that the drug delivered in this manner was well-tolerated and achieved concentrations in the respiratory tract orders of magnitude above its inhibitory concentration.

SARS-CoV-2, like many viruses that cause acute respiratory infections (ARIs), infects cells almost exclusively via the apical (luminal) side of the airway epithelium and also primarily buds from infected cells via the apical surface. Progeny virus must then travel through airway mucus to reach uninfected epithelial cells as the infection spreads from the upper respiratory tract (URT) to the lower respiratory tract (LRT) and the deep lung. Neutralizing monoclonal antibodies (mAbs) must therefore reach a high enough concentration in the airway lumen to effectively neutralize the virus and halt the infection.

mAbs distribute very poorly and slowly from the blood into the respiratory tract fluids, with concentrations in the airways that are orders of magnitude lower than those in the serum following intravenous (IV) or intramuscular (IM) administration. Despite these limitations, the clinical experience to date has shown that IV-administered mAbs against SARS-CoV-2 can be effective in treating infected individuals at high risk of severe COVID-19 when given early in the course of the infection, implying that sufficient amounts of mAb can distribute into the lung lumen. Nevertheless, high doses of mAb are generally required if given IV, reducing the number of treatment courses available from a given supply of drug. Delayed distribution into the lung also limits the treatment window for preventing severe COVID-19.

Although nebulization has been used to deliver protein therapeutics directly to the lungs, and direct inhaled delivery can quickly achieve far higher concentrations of drugs in the lungs than can be achieved by IV or IM administration, nebulized delivery from the mouth has not been previously shown to be effective for delivering a significant amount of therapeutic in the nasal passages.

As described herein, a nebulizer that generates a broad aerosol size distribution may be used to deliver drug throughout the entire respiratory tract, from the nasal turbinates in the URT, to conducting airways in the LRT, to the deep lung. This nebulized delivery may rapidly achieve high, inhibitory concentrations of mAb in the airway fluids along the entire respiratory tract. Nebulization also enables convenient self-dosing at home, reducing the burden on patients and on the healthcare infrastructure compared to systemic delivery.

A double-blind, placebo-controlled, first-in-human, ascending-dose pharmacokinetic and safety study was conducted in a Phase 1 unit in Melbourne, Australia. Eligible participants were enrolled sequentially into three cohorts: a single low dose cohort (30 mg), a single high-dose cohort (90 mg), and a multiple high-dose cohort (seven daily 90 mg doses). For each single dose cohort, a sentinel pair (with one active and one placebo recipient) was initially dosed, followed by a two-day safety monitoring period prior to the dosing of the remainder of the cohort. Advancing to subsequent cohorts was done after review of safety parameters seven days after final dosing of the preceding cohort. FIG. 3A shows a diagram of the process flow, study structure, and times of pharmacokinetic evaluations. Eligibility criteria required that participants be adults 18-55 years of age with a body-mass index of 18-32 kg/m² who were in good health as judged by medical history, physical exam, clinical chemistry and hematology assessments, electrocardiogram, forced expiratory volume in one second (FEV₁)≥90% predicted, and negative serology for HBsAg, HCV, and HIV antibodies. Participants were required to be non- or light smokers. The FEV₁ threshold was changed to ≥80% predicted after enrolling the first 7 participants. Participants were excluded for known or suspected symptomatic viral infection or signs of active pulmonary infection or pulmonary inflammatory conditions within 14 days of dosing initiation, a history of airway hyperresponsiveness, angioedema, anaphylaxis, or a positive alcohol breathalyzer test and/or urine drug screen for substances of abuse.

During recruitment of the 7 participants comprising the first single dose cohort, participants who had received a COVID-19 vaccine were excluded. However, due to rapidly increasing local vaccine availability and uptake, this criterion was modified to exclude only those vaccinated within two weeks of initial dosing, or those with plans to be vaccinated within two weeks after completion of dosing.

The primary endpoint for the trial was the safety and tolerability of IN-006. This was assessed by monitoring treatment-emergent adverse events, pre- and post-dose vital signs, ECG, FEV₁, SpO₂, hematology and chemistry safety blood tests, and physical examinations. Follow-up continued for 28 days, with assessments on the days indicated in FIG. 3B. Exploratory outcomes were drug levels in nasal fluid and serum pre-dose and at intervals post-dose. A randomization schedule was prepared using validated software (SAS) by statistical team members who had no responsibility for monitoring and data management of this study, with provisions for each sentinel pair to include one active and one saline placebo assignment, and for the overall ratio of active to placebo assignment of each cohort to be 3:1. The randomization code was held by unblinded pharmacy staff who prepared the doses in matching syringes with identical appearances for loading into the nebulizer by clinical staff.

IN-006 nebulized using a vibrating mesh nebulizer (Koninklijke Philips N.V.). Placebo participants received saline instead of IN-006. Participants were instructed to breathe in slowly through the nebulizer mouthpiece and to breathe out through their nose as described herein. Nasal fluid was obtained by rotating a flocked swab (Copans Cat. #56380CS01) for 10-15 seconds at mid-turbinate depth (4-5 cm). Sampling alternated between right and left nostrils during sequential sample collection timepoints. The amount of nasal fluid sample collected by each individual swab was determined by weighing the sample-containing swab and sample tube before and after it was incubated in buffer for extraction, rinsed, and oven dried. Nasal concentrations are therefore reported as ng IN-006 per gram of nasal fluid, which can be approximately interpreted as ng/mL. Sampling times for nasal fluid and serum are shown in FIG. 3B. Vital signs and FEV₁ were measured before nebulization and 15 and 30 minutes after completion of nebulization. Continuous variables were summarized using descriptive statistics including number of non-missing observations, mean, SD, median, minimum, and maximum values. Categorical variables were summarized with frequency counts and percentages. Placebo recipients in different cohorts were pooled. The safety analysis included all randomized participants who received any dose of study drug. The pharmacokinetic population included all participants who received any dose of IN-006.

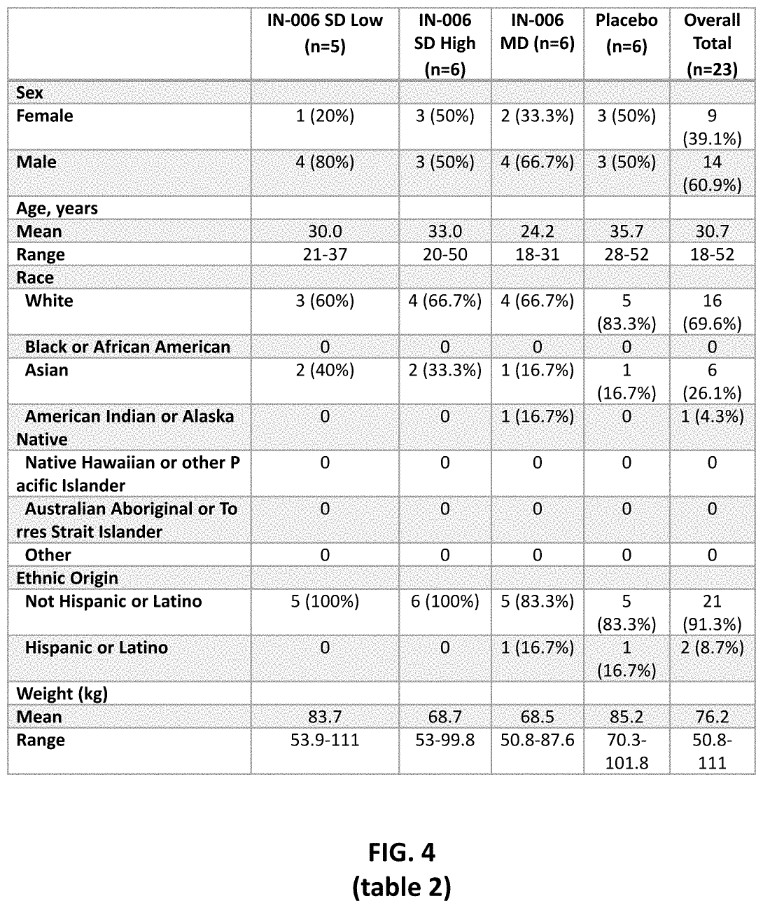

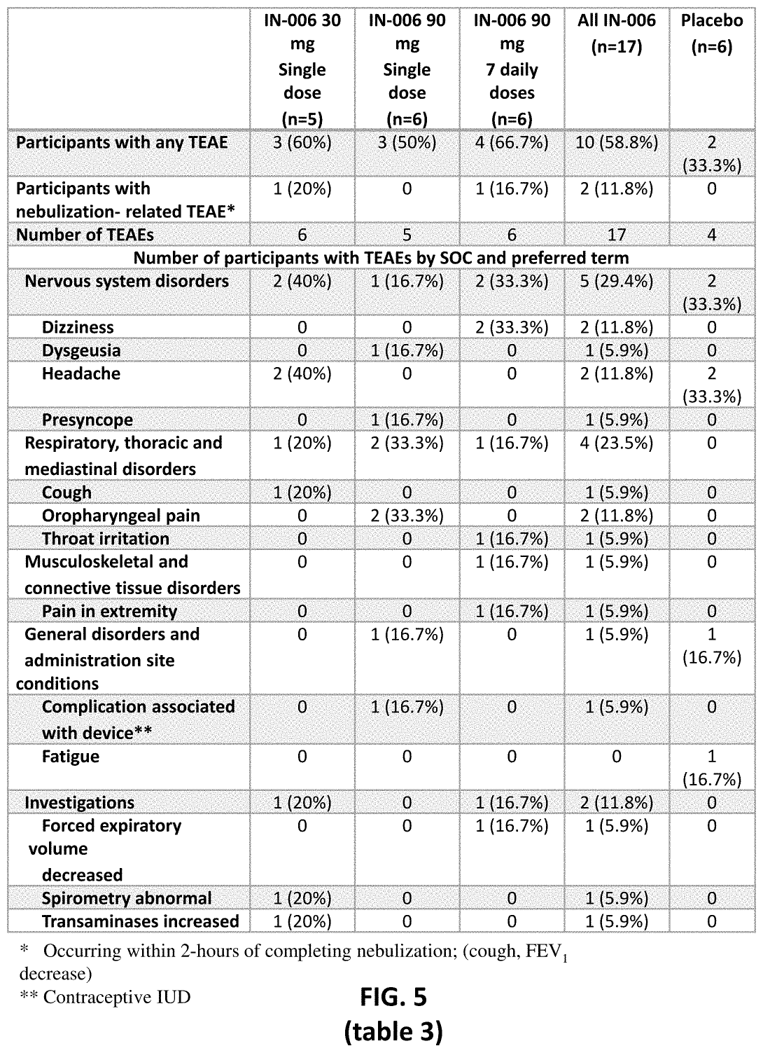

Of these participants, 17 were randomly assigned to receive IN-006, and 6 were randomly assigned to receive placebo. All 23 participants received their assigned treatment as intended and completed the final study visit on Study Day 29. Participant flow is diagrammed in FIG. 3A, and participant demographics are listed in FIG. 4 (Table 2). Treatment emergent adverse events (TEAEs) are listed in FIG. 5 (Table 3). Nebulization of IN-006 was well-tolerated and completed in an average of 6 minutes for the 90 mg dose (range 4-9 minutes). Eight (53.3%) of the 15 participants included in the single ascending dose cohorts experienced at least 1 TEAE (6 of 11 receiving IN-006, 2 of 4 receiving placebo). Among the 11 participants receiving IN-006, the most frequently reported TEAEs in the SAD cohorts were headache (2/11; 18.2%) and oropharyngeal pain (2/11; 18.2%). All but 1 TEAE were mild. One participant who received IN-006 low dose (30 mg) experienced a moderate event (increased transaminases on Day 29), which was not considered to be related to study drug by the investigator. Three (3/15; 20.0%) participants experienced at least 1 TEAE considered related to study drug by the investigator. These events included headache, cough, and oropharyngeal pain. All 3 related TEAEs were mild and resolved. There was no evidence of a dose-related effect.

In the multiple dose cohort, no TEAEs were reported in participants receiving placebo. Among the 6 participants receiving IN-006, 4 (66.7%) participants experienced at least 1 TEAE. The most frequently reported TEAE was dizziness (2/6; 33.3%). All but 1 TEAE were mild. One participant receiving IN-006 experienced a moderate event (pain in extremity), which was considered unlikely to be related to study drug by the investigator. Two (33.3%) participants experienced at least 1 TEAE considered related to study drug by the investigator. These drug related TEAEs were dizziness and decrease in FEV₁: the latter was noted 15 minutes after nebulization, was not associated with symptoms or abnormal vital signs, resolved within 15 minutes, and did not recur with subsequent doses. Both events were mild.

No severe TEAEs, SAEs, or TEAEs leading to discontinuations were reported in either the single dose or multiple dose cohorts. The most frequently reported TEAEs in participants receiving IN-006 across all three cohorts were headache (2/23; 11.8%) and oropharyngeal pain (2/23; 11.8%), both appearing only in the SAD cohorts and not in the multiple dose cohort. There were no unexpected safety signals.

Non-GLP ELISA-based bioanalytical methods were used for the analysis of IN-006 in human serum and human nasopharyngeal fluid. These assays used anti-idiotypic antibodies that bound IN-006 for capture and detection. Reference mAb and anti-ID antibodies, were used. The streptavidin-HRP conjugate of the anti-ID 18E5 was made using a Lightning Link® HRP Conjugation Kit (Abcam, Cat #ab103890). For the nasopharyngeal samples, the initial step involved extraction of IN-006 from nasopharyngeal swab prior to dilution to run on the ELISA. No extraction step was necessary for the serum samples. IN-006 standards, human serum samples, and quality control samples were diluted and added to a microtiter assay plate that had been coated with anti-ID 18D2, and the plate was then blocked. After washing, the streptavidin-HRP conjugated anti-ID 18E5 was then added, incubated, and washed. The plate was developed using 3,3′,5,5′,-tetramethyl benzidine (TMB, Thermoscientific, Cat #30429) and stopped with 2N sulfuric acid (Fisher, Cat #SA212-4). The absorbance was measured at 450 nm and 650. Data analysis was performed by creating a dose-response curve for the reference standards on each plate by plotting the signal in each well (y-axis) against the corresponding concentration of IN-006 (x-axis). The response plot was then fit with a 4-parameter logistic (4PL) non-linear regression model (using SoftMax software). The lower limit of quantitation (LLOQ) for the serum assays was 5 ng/mL, and the samples were diluted at least 5-fold, resulting in an effective LLOQ of 25 ng/mL. The LLOQ for the nasopharyngeal samples was variable (between 105-1,770 ng/g) due to the variability in the mass of collected samples and the extent of subsequent dilution of the nasopharyngeal fluid.

Nasal fluid pharmacokinetic data were available for all participants for all planned sampling time points. Mean IN-006 concentration in nasal fluid over time is plotted for each cohort. In each Cohort, Cₘₐₓ in the nasal fluid was observed at the first sampling point collected following dosing. In the single-dose Cohorts and multiple dose Cohort, a valid estimate of elimination rate was not able to be determined due to limited availability of samples with measurable drug concentrations (i.e., fewer than 3 concentration estimates after tₘₐₓ). Intranasal PK parameters of IN-006 were determined using Phoenix WinNonlin version 8.3. All times used in the calculation of pharmacokinetic parameters were the actual elapsed time from the most recent treatment administration, with the exception of pre-dose data which was given the nominal time of 0.00 h. The first samples were collected at 3 hours post-dose on Day 1 and then at 0.5 hours post-dose on Day 7, with the next sample at 22 hours post-dose on both days. The intranasal concentration was higher at 0.5 hours on Day 1 than at 3 hours post-dose on the same day, indicating that the concentration peaked soon after dosing, and was considerably reduced by 3 hours post-dose.

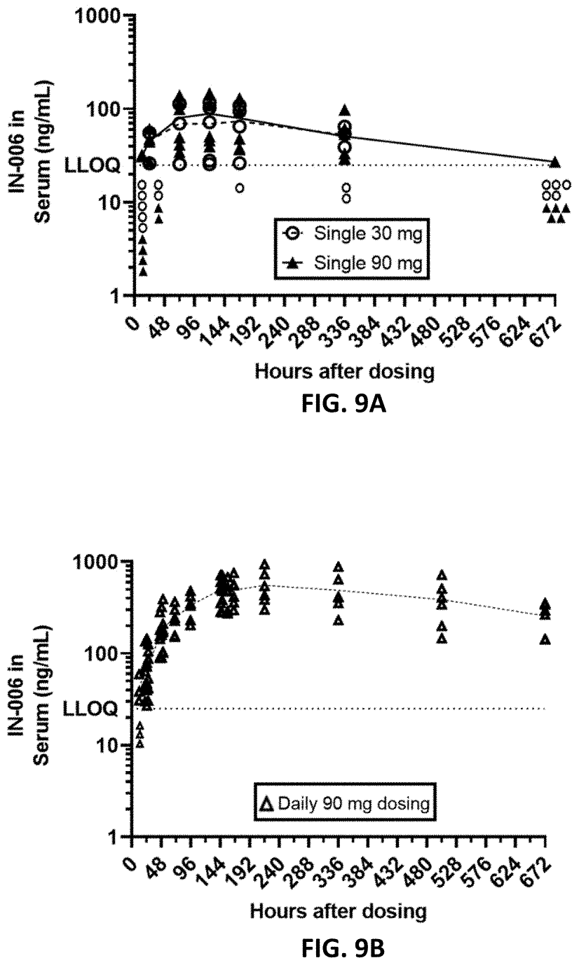

Serum pharmacokinetic data were available for all participants for all planned sampling time points. Mean IN-006 concentration in serum vs. time is plotted for each cohort in FIGS. 9A-9B. Key parameters of systemic exposure (tₘₐₓ, Cₘₐₓ, AUC₀₋ₜₗₐₛₜ, and t_{1/2}, also AUC_{0-infinity_obs} for MD) is presented in FIG. 7 (Table 5), showing median and range for tₘₐₓ and mean (% CV) for Cₘₐₓ and AUC. In the single-dose Cohorts, a valid estimate of elimination rate was able to be determined for only 2 of 5 participants in Cohort 1 and for only 2 of 6 participants in Cohort 2 due to limited availability of samples with measurable drug concentrations (i.e., fewer than 3 concentration estimates after tₘₐₓ). In the MD Cohort, however, estimates of elimination half-life could be generated for all participants, with a mean span of 0.97 and an R2 of suggesting that the collected samples described the terminal log-linear phase of elimination well, allowing the calculation of AUC_{0-infinity_obs}. For Cohorts 1 and 2, the last timepoint collected had many samples that were BLQ, decreasing the span for calculation of elimination rate constant and therefore decreasing certainty in AUC_{0-infinity} predictions, so AUCs were simply reported through tₗₐₛₜ in FIG. 7 (Table 5).

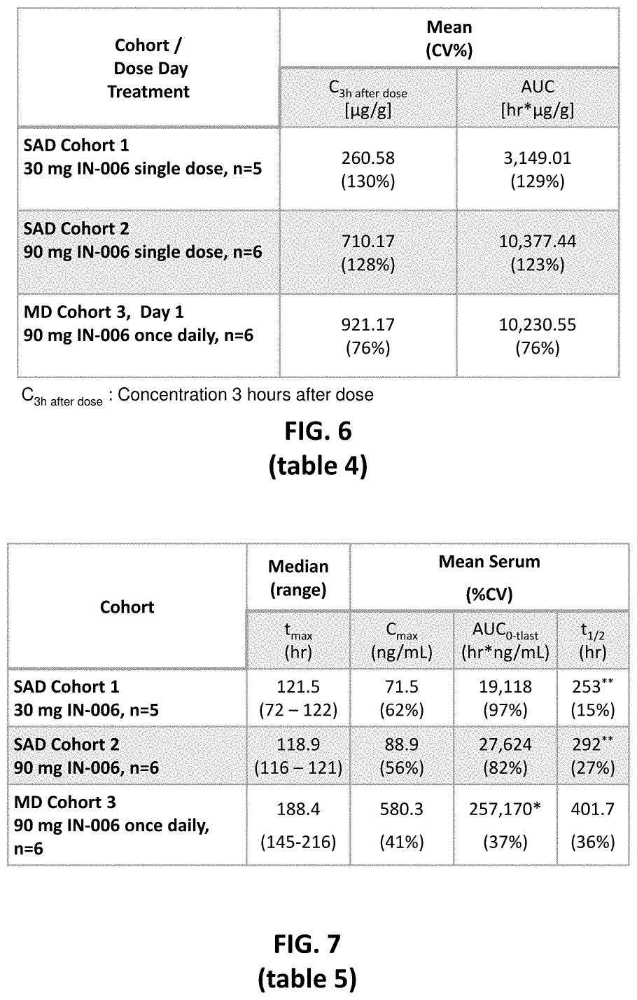

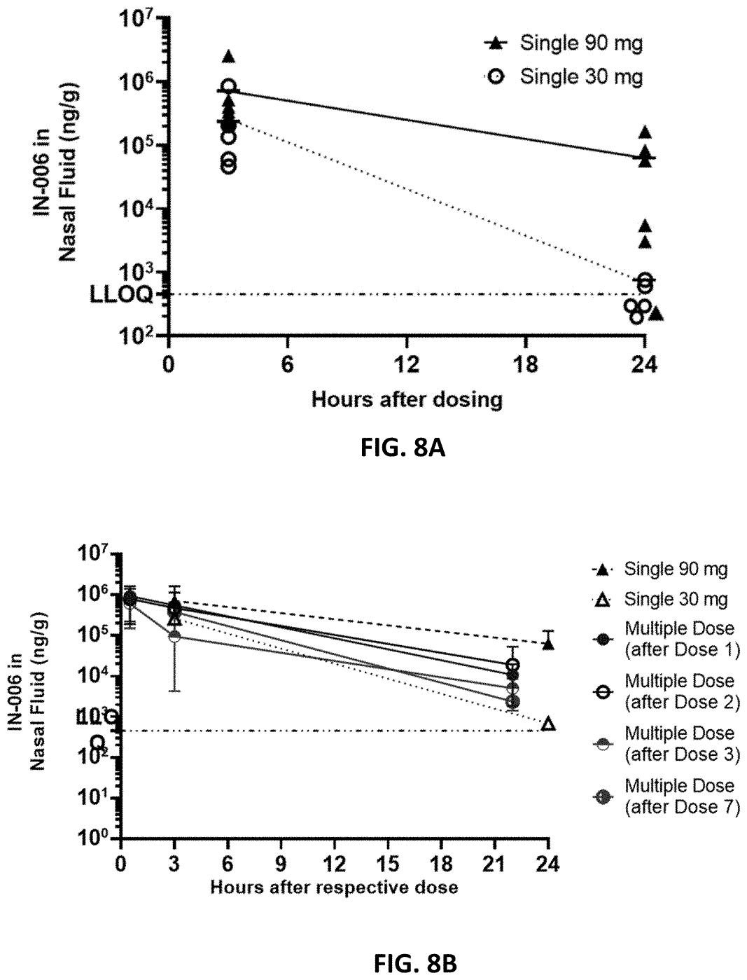

For single dose cohorts, the mean concentrations of IN-006 per gram of nasal fluid were 261 μg/g and 710 μg/g for the 30 mg and 90 mg dose, respectively, measured 3 hrs. after dosing; the difference in these values is consistent with a 3-fold increase in the dose administered, as shown in FIG. 6 (Table 4). In the multiple dose cohort, the repeated dosing provided additional opportunities for more nasal concentration measurements across more time points. The nasal concentrations measured 30 mins after dosing on Days 1, 2, and 3 averaged 773 μg/g, which was higher than the average concentration measured 3 hrs. after dosing (405 μg/g). This indicates that peak exposure occurred very shortly after dosing, and the nasal concentrations had appreciably reduced by 3 hours post-dose. There was minimal intranasal accumulation upon repeated dosing, as the concentrations of IN-006 measured 22 hers after a single dose were <2% of the concentrations immediately following dosing (FIGS. 8A-8B). The difference in nasal concentrations between 30 mins and 22 hrs. post-dose suggest the interval represented ˜6-7 half lives, and the difference between 30 mins and 3 hrs. post-dose was roughly half. Both are consistent with an intranasal half-life of roughly 3-4 hours, markedly longer than the timescale of mucociliary clearance transit time estimates of ˜5-15 minutes from saccharin transit time tests.

Serum concentrations of IN-006 were detectable by 12 hrs. following nebulization at the 90 mg dose and continued to rise through 120 hrs. after a single dose (cohorts 1 and 2), or through 216 hrs. following the first dose in those who received multiple doses (cohort 3). In the multiple dose cohort, Cₘₐₓ in the serum occurred following the final dose, indicating accumulation. The elimination half-life of IN-006 in the serum was estimated to be ˜253, 292, and 402 h in the 30 mg single dose, 90 mg single dose, and the 7 daily 90 mg dose cohorts respectively, comparable with the previously estimated elimination half-life of regdanvimab from the serum following intravenous administration (288 h). Although the serum concentrations of IN-006 were markedly lower than those in the nasal fluid (Serum Cₘₐₓ of 0.52 μg/mL, compared to 990 μg/mL in nasal fluid), they were still significantly greater than the IC₅₀ of IN-006 (˜0.01 μg/mL) against susceptible variants.

A longstanding dogma has been that it is highly challenging to stably nebulize mAbs, and that biologic drugs would be quickly eliminated from the respiratory tract either by systemic absorption, physical mucociliary clearance, or degradation by alveolar macrophages, making it difficult to sustain therapeutic concentrations. However, in this study, a therapeutic mAb (e.g., IN-006, a reformulation of regdanvimab for nebulized delivery at point of care), was safe and well tolerated in healthy adults. When inhaled orally, instructing the patient to inhale for (e.g., for 4 seconds or more) while exhaling more rapidly (e.g., for 3 seconds or less) through the nose, high concentrations of drug were recovered from nasal samples, as well as appreciable levels detected in the serum. The treatment was easily self-administered by participants and was completed within minutes, with minimal side effects. Surprisingly, the mean concentrations of IN-006 measured in nasal secretions, representing IN-006 deposited in the nasal passages after being exhaled from the lungs, were well above its IC₅₀, even 22-24 hours after dosing. In the multiple dose cohort that received seven daily 90 mg doses, subjects had a mean nasal fluid IN-006 concentration of 920 μg/mL 30 minutes after the initial dose and a mean concentration of 5.4 μg/mL 22 hours later, prior to receiving a second dose. The ability to maintain mAb concentrations that ranged from 3-7 orders of magnitude higher than the IC₅₀ for regdanvimab and other COVID mAbs against susceptible variants (˜4-20 ng/mL) strongly support a once-daily dosing regimen. Since SARS-CoV-2 infection and replication initiates in the upper respiratory tract, efficient delivery of IN-006 to the nasal passages suggests it may provide a highly effective treatment for mild to moderate COVID-19 that allows earlier resolution of the infection and reduced risk of progression to severe COVID.

Although mAbs have proven to be effective therapeutics for COVID-19, the necessity for administration by IV, IM, or SC routes has limited the scope of their use in clinical practice. The requirement for infusion centers and post-dosing observation for intravenous administration have severely limited the number of patients that have received treatment and greatly increased costs. IM injections, although shortening administration time, are limited by the volume that can be administered per injection (˜5 mL), which in turn limits the dose of mAb that can be dosed per injection and can be painful when maximum injection volumes are used. In contrast, nebulized delivery using a handheld nebulizer enables the convenience of at-home dosing and takes only minutes to complete. Furthermore, IV, IM, and SC routes provide mAb Cₘₐₓ to the airway lining fluid from the blood only after a delay of one or more days, and, even then, only achieve airway concentrations that are a fraction of the concentrations in plasma. For instance, in a recent clinical trial of the anti-influenza mAb CR6261 given as a single 50 mg/kg dose IV, the peak nasal concentration was not achieved until 2 days after IV infusion and the peak nasal concentration of 0.597 μg/mL was ˜10-fold lower than the concentrations we observed for IN-006 at the trough of our daily inhaled dosing (˜5.4 μg/mL), despite the much lower total dose of IN-006 compared to CR6261 (90 mg IN-006 vs. ˜2,000-4,000 mg CR6261). The increased convenience and more efficient pulmonary delivery may make inhalation the preferred route of mAb delivery for treating acute respiratory infections.

Although the example illustrated above is specific to the IN-006 therapeutic mAb described, similar or identical results may be seen with other nebulized therapeutic mAbs with respect to the levels of drug in the nasal passages, lungs and blood, as these results are generalizable to virtually any therapeutic mAb. This result is particularly surprising in mAbs that are selected or engineered for enhanced mucosal trapping (e.g., recombinant antibodies comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus), which may otherwise be expected to clear from the mucus more quickly and would not be expected to reach the levels of concentration in the nasal passages seen when inhaling the nebulized particles orally.

The methods described herein may be used with multiple therapeutic mAbs , e.g., neutralizing mAbs, to create a mAb cocktail. For example, these methods may be used to include mAbs that possesses potent binding activity against multiple variants e.g., of COVID, including Omicron BA.4/5.

Serious disease due to respiratory viruses (such as SARS-CoV-2) is often accompanied by the spread of the virus from the site of the initial upper respiratory tract infection to the deep lungs. Unfortunately, the exact timing of such spread is likely to be highly variable between individuals. Indeed, there is evidence suggesting viruses can reach the LRT even during the early stages of disease, around the time that symptoms emerge. Thus, we believe dosing to both the URT and LRT, rather than focusing exclusively on the URT (e.g., via nasal sprays), will be important to broaden the treatment window and reduce risk of COVID-induced pneumonia and hospitalization. While IN-006 levels in the LRT were not directly measured in this study, the appreciable serum concentrations and the delayed serum Tmax both strongly suggest we are efficiently delivering IN-006 into the LRT and the deep lung. Indeed, in a toxicokinetic multiple dose nebulization study of IN-006 in rats, IN-006 concentrations in airway fluid exceeded the serum concentrations by ˜100-fold. Efficient delivery into the LRT is a direct consequence of our design requirement for the vibrating mesh nebulizer. The droplet sizes generated by the nebulizer (the fine particle fraction, i.e. droplets <5 um, and particularly those <2.5 um) were intentionally selected to deliver a portion of the mAbs throughout the LRT and deep lung. Furthermore, the fact that we observed a slow steady rise of serum concentrations in single dose cohorts over ˜4 days, and peak serum concentrations in the multiple dose cohort over ˜9 days (or 2 days after last dose), implies that we are sustaining high levels of IN-006 in the deep lungs for at least 2-4 days after a single dose (or after the final dose). Assuming a ratio of 100:1 of antibody concentrations in the airway fluid:serum, the mean serum concentration of 550 ng/mL at Day 9 should translate to pulmonary concentrations on the order of 55 μg/mL, which is >3 orders of magnitude above the IC₅₀, and comparable to the serum concentrations achieved with some IV/IM-dosed mAbs. The very high mAb levels sustained relative to the intrinsic activity of the mAb (IC₅₀) may continue to provide effective treatment against variants, even in the presence of appreciable genetic drift, and may reduce the risk of viral escape. This also suggests that shorter durations of therapy may afford appreciable protection against hospitalization.

Despite significant extrapulmonary manifestations of severe COVID-19, and despite frequent detection of SARS-CoV-2 RNA in blood, infectious SARS-CoV-2 is rarely detected in the blood of infected patients, suggesting that extrapulmonary disorders are in many cases caused by indirect factors such as the inflammatory response rather than extrapulmonary viral infection. Nonetheless, mean serum levels of IN-006 achieved after nebulized delivery were in excess of its IC₅₀ by at least one order of magnitude.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of delivering particles of delivering nebulized particles of a drug agent to a patient's nasal passages, the method comprising:
operating a nebulizer containing the drug agent to continuously form particles containing the drug agent;
holding a mouthpiece of the nebulizer in the mouth with lips sealed over the mouthpiece; and
repeatedly inhaling for 4 seconds or longer from the nebulizer through the mouth followed by exhaling only through the nose for 3 seconds or less.

2. The method of claim 1, wherein nebulized particles including the drug agent are delivered in an approximately equivalent surface concentration between the patient's nasal passages and lungs.

3. The method of claim 1, wherein inhaling for 4 seconds or longer comprises inhaling for between 4-10 seconds.

4. The method of claim 1, wherein exhaling only through the nose for 3 seconds or less comprises exhaling only through the nose for 2 seconds or less.

5. The method of claim 1, further comprising triggering a first indicator for the period of inhalation, wherein the first indicator is triggered for 4 seconds or longer and triggering a second indicator for the period of exhalation wherein the second indicator is triggered for 3 seconds or less.

6. The method of claim 1, wherein operating the nebulizer to continuously form particles containing the agent comprises forming particles of average particle or droplet size in the range from about 0.1 to about 200 microns.

7. The method of claim 1, wherein operating the nebulizer to continuously form particles containing the agent comprises forming particles of average particle or droplet size in the range from about 2 to 7 microns.

8. The method of claim 1, wherein the drug agent is a recombinant antibody comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

9. The method of claim 1, wherein the drug agent comprises a recombinant antibody comprising a human or humanized Fc region, wherein the recombinant antibody comprises a population of antibodies in which at least 40% comprise an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

10. A method of delivering an approximately equivalent surface concentration of nebulized particles of a drug agent between a patient's nasal passages and lungs, the method comprising:
operating a nebulizer containing the drug agent to continuously form particles containing the drug agent;
holding a mouthpiece of the nebulizer in the mouth with lips sealed over the mouthpiece; and
repeating one or more cycles of:
inhaling, for an inhalation period of 4 second or longer, particles containing the drug agent through the mouth for the inhalation period, and
exhaling, for an exhalation period of 3 seconds or shorter, through the nose but not the mouth for the exhalation period,
wherein an approximately equivalent surface concentration of the particles containing the drug agent is deposited between the patient's nasal passages and lungs.

11. A method of delivering an approximately equivalent surface concentration of nebulized particles of a drug agent between a patient's nasal passages and lungs, the method comprising:
operating a nebulizer containing the drug agent to continuously form particles containing the drug agent;
holding a mouthpiece of the nebulizer in the mouth with lips sealed over the mouthpiece; and
repeating one or more cycles of:
indicating, with a first indicator, an inhalation period of 4 second or longer, to guide the patient in inhaling particles containing the drug agent through the mouth for the inhalation period, and
indicating with a second indicator, an exhalation period of 3 seconds or shorter, to guide the patient in exhaling through the nose but not the mouth for the exhalation period,
wherein an approximately equivalent surface concentration of the particles containing the drug agent is deposited between the patient's nasal passages and lungs.

12. The method of claim 11, wherein the approximately equivalent surface concentration of the particles containing the drug agent comprises a surface concentration of between about 10:1 and 1:10 between the patient's nasal passages and lungs.

13. The method of claim 11, further comprising triggering the first indicator at a start of inhalation by the patient.

14. The method of claim 11, further comprising triggering the second indicator at a start of exhalation by the patient.

15. The method of claim 11, wherein the first indicator comprises illuminating a first LED.

16. The method of claim 11, wherein the second indicator comprises illuminating a second LED.

17. The method of claim 11, wherein the inhalational period is between 4-10 seconds.

18. The method of claim 11, wherein the exhalation period is 2 seconds or less.

19. The method of claim 11, wherein operating the nebulizer to continuously form particles containing the agent comprises forming particles of average particle or droplet size in the range from about 0.1 to about 200 microns.

20. The method of claim 11, wherein operating the nebulizer to continuously form particles containing the agent comprises forming particles of average particle or droplet size in the range from about 2 to 7 microns.

21. The method of claim 11, wherein the drug agent is a recombinant antibody comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

22. The method of claim 11, wherein the drug agent comprises a recombinant antibody comprising a human or humanized Fc region, wherein the recombinant antibody comprises a population of antibodies in which at least 40% comprise an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.
